(19) United States
(12) Patent Application Publication
(10) Pub. No.: US 2015/0265470 A1
(43) Pub. Date: Sep. 24, 2015

(54) Methods and Systems for Performing a Capsulotomy

(71) Applicants: Carl Zeiss Meditec AG, Jena (DE); Carl Zeiss AG, Oberkochen (DE)

(72) Inventors: Marco WILZBACH, Stuttgart (DE); Artur HOEGELE, Oberkochen (DE); Michael Stefan RILL, Jena (DE); Joachim STEFFEN, Westhausen (DE); Frederik SIEGMANN, Berlin (DE); Johannes Heinrich KINDT, Weimar (DE)

(21) Appl. No.: 14/661,245

(22) Filed: Mar. 18, 2015

Related U.S. Application Data
(60) Provisional application No. 61/968,653, filed on Mar. 21, 2014.

(51) Int. Cl. A61F 9/008

(52) U.S. Cl. CPC A61F 9/00825; A61F 2009/0087; A61F 2009/00889

(57) ABSTRACT

A method of performing a capsulotomy comprises applying a light-absorbing agent to a lens capsule of an eye 1; illuminating the lens capsule using a first light beam and recording an image of the lens capsule using a camera 43; analyzing the recorded image and determining whether the light absorbing agent has been correctly applied to the lens capsule; if it is determined that the light absorbing agent has been correctly applied to the lens capsule, directing a shaped second light beam 19 onto a predetermined line on the lens capsule in order to modify a structure of a tissue of the lens capsule. The first light beam can be generated by defocusing the second light beam using a lens 37.

### CROSS-REFERENCES TO RELATED APPLICATIONS

The present application claims priority of Provisional Patent Application No. 61/968,653, filed Mar. 21, 2014 in the United States of America, the entire contents of which are incorporated by reference herein.

### FIELD

The present invention relates to cataract surgeries.

### BACKGROUND

In a cataract surgery, the opacified natural lens is removed while leaving certain structures of the elastic lens capsule intact to allow implantation and retention of an intraocular lens (IOL). One essential step in such surgery is commonly referred to as capsulorhexis and includes providing a hole in the lens capsule to permit removal of the nucleus and cortex of the natural lens. Moreover, the intraocular lens is inserted into the lens capsule through this hole. It is currently believed that the hole ideally has a circular shape of a size adapted to the diameter of the intraocular lens. Traditionally, the capsulorhexis is performed using a surgical tool such as forceps or needle. This procedure is difficult and requires an experienced surgeon in order to reliably obtain good results.

It has been considered to use a high-power femtosecond laser in order to modify and weaken the tissue of the lens capsule along a circular path defining the hole to be formed in the lens capsule. It is subsequently possible to reliably remove the tissue in the interior of the circular path, even for a less experienced surgeon. However, the high-power femtosecond laser is very expensive.

The process of weakening the tissue of the lens capsule by a laser beam is referred to as capsulotomy.

It has recently been suggested to apply a light absorbing agent to the anterior lens capsule and to use a lower-power CW laser to scan a tissue modifying focused laser beam along a path defining the hole to be formed in the lens capsule. The light absorbing agent absorbs the laser light to cause a local thermal effect at the tissue of the lens capsule which modifies the tissue such that it is weakened. The tissue in the interior of the closed path can be removed subsequently.

As far as it is known to the inventors, this method has not yet been applied to a human eye due to security concerns. For example, the laser light used for modifying the tissue of the lens capsule should not damage the retina of the eye, and it should be prevented that tissue adjacent to the lens capsule receives the light absorbing agent since also those tissues could be modified by the laser beam.

### SUMMARY

The present invention has been made taking the above considerations into account.

Embodiments of the present invention provide methods and systems for performing a capsulotomy in which a light absorbing agent is applied to the lens capsule and a shaped light beam is directed onto a line on the lens capsule in order to modify a structure of a tissue of the lens capsule.

According to some embodiments, a method of performing a capsulotomy comprises: applying a light-absorbing agent to a lens capsule; illuminating the lens capsule using a first light beam and recording an image of at least the lens capsule; analyzing the recorded image and determining whether the light absorbing agent has been correctly applied to the lens capsule; and if it is determined that the light absorbing agent has been correctly applied to the lens capsule, directing a shaped second light beam onto a predetermined line on the lens capsule in order to modify a structure of a tissue of the lens capsule.

The lens capsule is formed of a transparent tissue which normally does not absorb substantial amounts of light. Therefore, it is difficult to modify the lens tissue by illumination with a condensed light beam since only a very low amount of the illuminated light is absorbed in the tissue such that a tissue modifying local thermal effect is not achieved. However, since the light absorbing agent is applied to the lens capsule, the light absorbing agent is located in immediate proximity of the lens capsule when the directing of the shaped light beam onto the predetermined line on the lens capsule is performed. The light absorbing agent absorbs a substantial amount of the light of the shaped second light beam such that a local thermal effect is caused at the tissue of the lens capsule such that the structure of that tissue is modified such that the capsulorhexis can be reliably performed by the surgeon.

The inventors have found that this procedure can be successful if one or more of the following desires are fulfilled: the retina of the eye should not be damaged by the focused light beam; apart from the lens capsule, other tissues of the anterior portion of the eye, such as the cornea, should not receive the light absorbing agent in order to prevent that also those tissues are accidentally modified by the focused light beam; the tissue of the lens capsule should have received a required amount of the light absorbing agent at all locations of the predetermined line; the amount of energy per unit area deposited by the shaped second light beam at each location of the predetermined line should be sufficiently high to achieve the desired modification of the tissue but should not substantially exceed this amount in order to prevent undesired modifications of the tissue of the lens capsule and adjacent tissues; the shaped second light beam should be shaped such that it illuminates a line exactly coinciding with the predetermined line also when the patient inadvertently moves his eye during the illumination; the shaped second light beam should be converged as near as possible at the tissue of the lens capsule at all locations of the illuminated line; the outer surface of the lens capsule should be contacted by a medium, such as air, water or a viscoelastic medium, when the light absorbing agent is applied or the illumination with the shaped second light beam is performed; and the desired result of tissue modification is achieved.

In order to improve the method with respect to at least some of the above desires, the lens capsule is illuminated with the first light beam and the image of the lens capsule is recorded subsequent to applying the light absorbing agent to the lens capsule. The recorded image is analyzed, and it is determined whether the light absorbing agent has been correctly applied to the lens capsule. Only if the correct applications of the light absorbing agent to the lens capsule is confirmed, the directing of the shaped second light beam onto the predetermined line is performed. If it is not possible to confirm the correct application of the light absorbing agent, the directing of the shaped second light beam can be prevented.

The directing of the shaped light beam onto the predetermined line can be achieved in various ways. For example, the illumination of the predetermined line as a whole can be performed simultaneously or sequentially. According to some embodiments, the light beam is shaped such that it forms a focus on the lens capsule, and the beam is scanned such that the focus is continuously moved along a scanning path coinciding with the predetermined line, or the focus is stepwise moved to each of a plurality of locations distributed along such scanning path, wherein the focus is maintained at each location for a suitable time in order to deposit a necessary amount of energy at each location. According to other embodiments, the light beam is shaped such that it is converged at the lens capsule and simultaneously covers the whole predetermined line or at least a portion of the predetermined line.

The first and second light beams can be generated by different light sources. The first and second light beams may include light of different wavelength ranges which can be even non-overlapping wavelength ranges. According to exemplary embodiments, the first light beam includes light of a wavelength which is equal to a wavelength of light included in the second light beam. This means that the wavelength ranges of the first and second light beams are at least overlapping wavelength ranges. Moreover, the wavelength ranges of the light of the first and second light beams can be identical. According to some exemplary embodiments, the first and second light beams are generated by a same light source. This light source can be a laser light source and, in particular, a CW laser light source. While the second light beam used for the illumination of the predetermined line is a converged or focused light beam, the first light beam used to illuminate the lens capsule for recording the image of the lens capsule desirably illuminates the whole lens capsule or an extended portion of the lens capsule. This can be also achieved by scanning the first light beam across the whole surface of the lens capsule if the first light beam is also a focused light beam. For example, an exposure time for the recording of the image can be selected sufficiently long such that the whole surface of the lens capsule can be scanned by the first light beam during the exposure for the image recording.

According to exemplary embodiments, the first light beam is generated by defocusing the second light beam. The second light beam, which is converged onto the lens capsule during the scanning can be defocused such that the light of the light beam illuminates the whole surface of the lens capsule for achieving the necessary illumination during the recording of the image. The defocusing can be achieved, for example, by inserting a diverging lens into the beam path of the second light beam or by removing a focusing lens from the beam path of the second light beam.

According to some embodiments, the light absorbing agent is a dye. A dye is a substance that has an affinity to the substrate to which it is applied, wherein the affinity is a property by which molecules of the dye form compounds with molecules of the tissue of the lens capsule. According to some particular embodiments herein, the dye is a fluorescent dye such that illumination of the dye with light of a certain wavelength results in emission of fluorescent light from a different wavelength range from the illuminated dye. According to some particular examples, the dye is selected such that the fluorescent emission of the dye is different for portions of the dye having formed a compound with the tissue from portions of the dye having not formed a compound with the tissue such that these two states of the dye can be discriminated based on the fluorescent emission of the dye. According to an exemplary embodiment herein, the light absorbing agent is Trypan blue. When the light absorbing agent is Trypan blue it can be advantageous for the first or second light beams to include light of a first wavelength range from 400 nm to 600 nm, and to record the image using light of a second wavelength range from 600 nm to 800 nm. For discriminating the two states of the fluorescent dye based on the fluorescent emission it can be advantageous if light outside the second wavelength range contributes to the intensity of the recorded image by less than 20% or does substantially not contribute to the recorded image.

The analyzing of the recorded image may include an automatic image processing. Moreover, the intensity of the second light beam can be controlled based on the recorded image during the scanning of the focus second light beam along the predetermined path. If it is assumed that the detected light intensity in the recorded image depends on a density of the light absorbing agent at locations of the lens capsule corresponding to locations within the image, it can be also assumed that the absorbed amount of light of the second light beam and the corresponding thermal effect depend on the density of the light absorbing agent applied to locations of the lens capsule.

According to some further embodiments, a method of performing a capsulotomy comprises: applying a light-absorbing agent to a lens capsule; scanning a focused light beam along a predetermined line on the lens capsule in order to modify a structure of a tissue of the lens capsule; detecting light of a predetermined wavelength range originating from the lens capsule during the scanning; and controlling at least one of an intensity of the focused light beam, a scanning speed of the focused light beam along the predetermined path and a focus position of the focused light beam in a longitudinal direction of the focused light beam based on an intensity of the detected light.

In order to modify the tissue of the lens capsule as desired, it is necessary to deposit a required amount of energy in or at the tissue by the absorption of the light of the focused light beam by the light absorbing agent. The amount of energy deposited per unit area and unit time depends on the amount of light absorbing agent located in or at the tissue since the density of the light absorbing agent applied to the lens capsule is generally not constant across the whole surface of the lens capsule or along the predetermined line, it is desirable to control parameters of the scanned light beam such that the desired amount of energy per unit area is deposited along the scanned line. For this purpose, light originating from the lens capsule during the scanning is detected. The intensity of this detected light is indicative of the amount of energy currently deposited by the scanning light beam in the tissue. The detected intensity can be processed, and one or more parameters of the scanning light beam can be controlled based on the detected light intensity. It is possible to detect only or substantially only light within a predetermined wavelength range. This wavelength range can be adapted to the light absorbing agent used and to the wavelength of the scanning light beam. Moreover, if the light absorbing agent is a fluorescent dye, this wavelength range can be selected such that only or substantially only the fluorescent light generated by the incident scanning light beam is detected.

The parameters of the scanning light beam which can be controlled based on the detected light comprise the intensity of the focused light beam, the scanning speed of the focused light beam along the predetermined path, and the focus position of the focused light beam in the longitudinal direction of the focused light beam.

According to some embodiments, the intensity of the focused light beam is reduced when the intensity of the detected light increases. In particular, the controlling of the intensity of the focused light beam may include preventing that the focused light beam reaches the lens capsule when the intensity of the detected light is below a threshold. If the intensity of the detected light is below a threshold, this is an indication that the light beam is currently scanning a portion of the lens capsule which has not received light absorbing agent such that the desired thermal effect can not be achieved while the light transmitted through the lens capsule may damage other tissue, such as the retina.

According to further exemplary embodiments, the scanning speed of the focused light beam is increased when the intensity of the detected light increases.

According to further exemplary embodiments, the focus position of the focused light beam is adjusted such that the focus of the focused light beam is located on or close to the lens capsule such that a high amount of energy is deposited per unit time and unit area.

According to further embodiments, a method of performing a capsulotomy comprises: applying a light-absorbing agent to a lens capsule; using beam supplying optics to generate a shaped light beam of a first intensity and to direct the shaped light beam of the first intensity onto a selected line relative to the beam supplying optics such that the shaped light beam of the first intensity is incident on an anterior portion of an eye; generating an image of the anterior portion of the eye wherein a line illuminated by the shaped light beam of a first intensity is detectable based on the generated image; at least one of positioning the beam supplying optics relative to the eye and modifying a parameter of the selected line based on the detected image such that the illuminated line coincides with a predetermined line on the lens capsule; and then using the beam supplying optics to generate a shaped light beam of a second intensity greater than the first intensity and to direct the shaped light beam of the second intensity onto the selected line relative to the beam supplying optics in order to modify a structure of a tissue of the lens capsule along the predetermined line on the lens capsule.

This method is useful for achieving a line illuminated by the shaped light beam of the second intensity modifying the tissue such that the actually illuminated line conforms with the desired illuminated line. The actual illuminated line may depend on the shape of the lens capsule of the individual eye and a position of the beam supplying optics relative to the lens capsule. Therefore, the illuminated line generated by the beam supplying optics has to be adapted to the situation under which the method is performed. The first intensity of the shaped light beam is selected such that the shaped light beam incident on the lens capsule does substantially not modify the tissue of the lens capsule. The second intensity of the shaped light beam, is, however, selected such that the tissue of the lens capsule is modified by the incident beam of light. The shaped light beam of the first intensity is used to align the actually illuminated line to the desired illuminated line, while the light beam of the second intensity is used to modify the tissue along the aligned illuminated line which substantially coincides with the desired illuminated line.

The shaped light beam of the first light intensity is incident on the tissue of the lens capsule to which the light absorbing agent has been applied. The light absorbing agent absorbs light of the shaped light beam of the first intensity and does also reflect a certain amount of this light. This light is incident onto the camera configured to record the image of the lens capsule and makes the actual illuminated line visible within the recorded image. The visible illuminated line of the recorded image can be compared with the desired or predetermined line on the lens capsule and one or more parameters influencing the illuminated line on the lens capsule can be modified based on this comparison. The at least one parameter includes at least one of a lateral position of the selected line relative to the beam supplying optics, a longitudinal position of the selected line relative to the beam supplying optics, i.e. a distance of the beam supplying optics from the lens capsule, and a lateral extension of the selected line. The selected line can be a circle or an ellipse, and the at least one parameter may include a diameter of the circle or ellipse, respectively, i.e. it's lateral extension, and a lateral position relative to the beam supplying optics.

It is also possible to use an ocular for generate the image when the light beam of the first intensity includes light from the visible portion of the spectrum.

According to some embodiments, a representation of the predetermined line is generated in the image. The representation of the predetermined line can be visually compared with the actual line which is visible in the recorded image, such that an operator receives a visual feedback when the parameters defining the illuminated line are changed by the operator. It is, however, also possible that the changing of the parameters of the illuminated line occurs automatically. According to particular embodiments, the representation of the predetermined line comprises a pair of adjacent spaced apart lines extending on both sides of the predetermined line. The actual illuminated line will then correspond to the desired line if the parameters are modified such that the actual line visible in the recorded image extends exactly between the pair of adjacent lines.

According to some embodiments, a system for performing a capsulotomy comprises: beam supplying optics comprising a light source, a beam shaping optics configured to shape a light beam from light emitted from the light source such that a shaped light beam is directed onto a lens capsule, and a beam intensity modifier; a camera configured to record an image of the lens capsule; a controller having two modes of operation: an alignment mode and a tissue modifying mode; wherein, in the alignment mode, the controller is configured to control the beam intensity modifier such that the light beam has a first intensity, wherein the first intensity is selected such that the shaped light beam of the first intensity does substantially not modify a tissue of the lens capsule, to control the beam shaping optics to direct the shaped beam having the first intensity onto a line on the lens capsule, wherein the line is defined by at least one parameter having a selected value, to control the camera to record an image of the lens capsule, to analyze the recorded image and to identify a line illuminated by the shaped light beam having the first intensity in the image, to compare the identified illuminated line with a predetermined line on the lens capsule, and to modify the value of the at least one parameter based on the comparison; wherein, in the tissue modifying mode, the controller is configured to control the beam intensity modifier such that the light beam has a second intensity greater than the first intensity, and to control the beam scanner to direct the shaped beam onto a the line on the lens capsule, wherein the line is defined by the at least one parameter having the modified value, in order to modify a structure of the tissue of the lens capsule along the line on the lens capsule.

The beam supplying optics can be mounted relative to the lens capsule by a stand including at least one actuator configured to displace the beam supplying optics relative to the lens capsule, and the controller can be configured to control the actuator based on the comparison in the alignment mode.

### BRIEF DESCRIPTION OF THE DRAWINGS

The forgoing as well as other advantageous features of the disclosure will be more apparent from the following detailed description of exemplary embodiments with reference to the accompanying drawings. It is noted that not all possible embodiments necessarily exhibit each and every, or any, of the advantages identified herein.

FIG. 1 is a schematic diagram illustrating a system for performing a capsulotomy according to a first embodiment;

FIG. 2 is a flowchart illustrating a method of performing a capsulotomy according to a second embodiment;

FIG. 3 is a flowchart illustrating a method of performing a capsulotomy according to a third embodiment; and

FIG. 4 is a flowchart illustrating a method of performing a capsulotomy according to a fourth embodiment.

### DETAILED DESCRIPTION OF EXEMPLARY EMBODIMENTS

In the exemplary embodiments described below, components that are alike in function and structure are designated as far as possible by alike reference numerals. Therefore, to understand the features of the individual components of a specific embodiment, the descriptions of other embodiments and of the summary of the disclosure should be referred to.

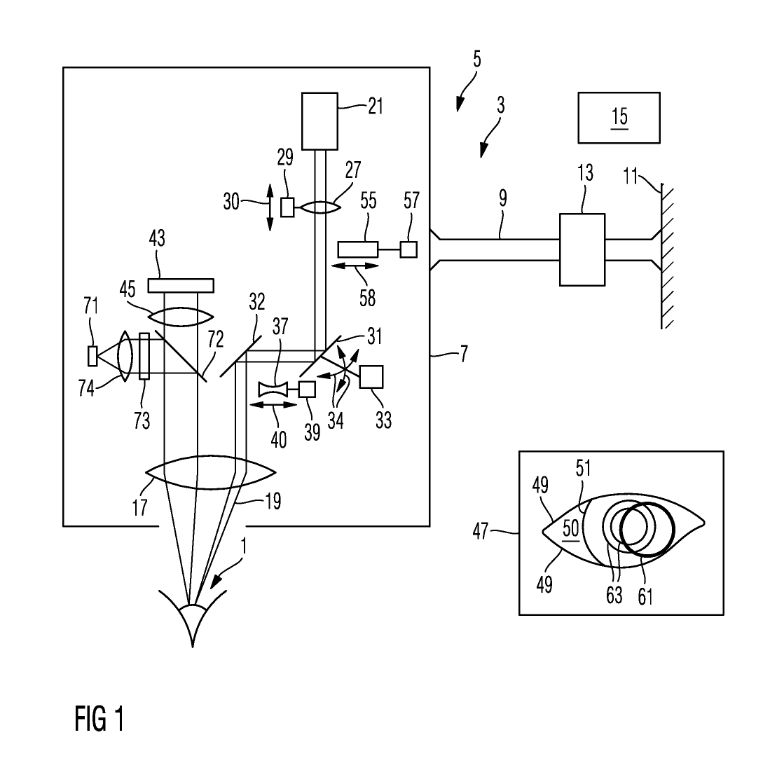

FIG. 1 schematically illustrates a system for performing a capsulotomy. An eye of a patient is schematically illustrated at 1. It is assumed that the eye 1 of the patient has developed a cataract such that the natural lens of the eye has to be replaced by an implant which is referred to as an intraocular lens. It is also possible, that the eye has an improper optical power, resulting in an eye sight deficiency, and an intraocular lens will be implanted to improve the visual faculty of the eye (refractive lens exchange).

For this purpose, a capsulorhexis has to be performed which includes to form a hole in the lens capsule. The natural lens is removed through this hole, and the intraocular lens is inserted into the lens capsule via this hole. The system 3 is used to assist in the capsulorhexis. In particular, the system emits a focused light beam which is scanned along a predetermined path on the lens capsule in order to modify the tissue of the lens capsule such that it is weakened. The scanning path defines the hole, and the tissue material in the interior portion of the scanning path can be easily removed subsequent to the weakening, thus forming the hole. The illustrated method of performing the capsulorhexis include application of a light absorbing agent to the lens capsule. Background information relating to such method can be obtained from U.S. Pat. No. 8,409,182 B2, the full disclosure of which is incorporated herein by reference.

The light absorbing agent can be a fluorescent dye. In particular, the light absorbing agent can be a fluorescent dye allowing to discriminate dye adhering to a substrate from dye not adhering to the substrate. In the illustrated embodiments, the light absorbing agent is Trypan blue. Trypan blue is a blue acid dye that contains two azo chromophores available from Sigma-Aldrich, St. Louis, Mo., USA. It is a large, hydrophilic, tetrasulfonated dye. When Trypan blue binds to proteins the resulting complex emits red fluorescence.

The system 3 comprises an optics part 5 which may be mounted in a housing 7, wherein the housing 7 is carried by a stand 9 which can be fixed at a roof, floor or wall 11 of a room. Since the patient with his head and eye rests on a support which is fixed relative to the room, the optics portion 3 is mounted relative to the eye 1 by the stand 9. The stand 9 includes an actuator 13 configured to change a position of the optics portion relative to the eye. The actuator 13 is controlled by a controller 15 of the system 3. The controller 15 may comprise one or more pieces of electronic circuits including a processor and memory which can be located within the housing 7 or outside of the housing 7 and even distant from the optics portion. For example, the controller 15 can be embodied as a computer running software connected to the various components of the system 3.

The optics portion comprises a main lens 17 which is used to direct light to the eye and to receive light from the eye. In the present embodiment, plural different types of light are directed towards the eye, and plural different types of light are received from the eye 1, and all these types of light traverse the same lens 17. It is, however, possible to provide plural such front lenses for different types of light directed towards the eye or received from the eye. The optics portion can be configured as a separate module which is carried by a separate stand or attached to another system used in the surgery. For example, the optics portion can be attached to a surgical microscope used by the surgeon. Moreover, it is possible that the optics portion is integrated with a surgical microscope such that components of the integrated system are used to perform functions of the optics portion as described in more detail below and functions of the surgical microscope.

One type of light directed towards the eye 1 is a focused light beam 19 used to modify the tissue of the lens capsule such that the capsulorhexis can be performed. The light of the focused light beam 19 is generated by a light source 21. In the illustrated example, the light source 21 is a CW laser. Other suitable light sources can be used as well. The light emitted by the laser source 21 is collimated by a lens 27 such that a focus of the light beam 19 is generated on the lens capsule of the eye 1. A position of the lens 27 along the beam path of the beam 19 can be changed by an actuator 29 as indicated by an arrow 30 in FIG. 1. The actuator 29 is controlled by the controller 15 in order to change a position of the focus of the beam 19 in the longitudinal direction of the beam 19. The actuator 29 can be operated before modifying the tissue of the lens capsule by scanning the focus of the beam 19 along a predetermined path on the lens capsule for adapting the scanned beam to different distances of the optics portion 3 relative to the eye, and the actuator can also be operated during the scanning if the distance of the lens capsule changes along the scanned path due to a curvature of the lens capsule, for example.

The beam 19 is reflected by two mirrors 31 and 32. In the present example, the mirror 32 is stationary while the mirror 31 is a scanning mirror operated by an actuator 33 configured to change an orientation of the mirror 31 in two independent directions as indicated by arrows 34 in FIG. 1. It is also possible that both mirrors 31 and 32 are scanning mirrors wherein an orientation of the mirror 31 can be changed by an actuator in one direction while an orientation of the mirror 32 can be changed by a further actuator in another direction. Moreover, additional mirrors can be provided and mirror 32 can be even omitted if mirror 31 is a scanning mirror allowing scanning in two independent directions.

As far as illustrated above, the light generated by the laser light source 21 is shaped such that the beam 19 forms a focus at or close to the lens capsule in order to modify the tissue of the lens capsule. A diverging lens 37 is mounted relative to the beam path of beam 19 by an actuator 39 such that the diverging lens 37 is selectively located outside of the beam 19 or within the beam 19 as indicated by an arrow 40. When the diverging lens 37 is located outside of the beam path of the beam 19, the focus is formed at or close to the lens capsule. When the diverging lens 37 is inserted into the beam path, the light of the beam 19 is diverged such that the focus is not formed on the lens capsule and the light is distributed across the whole anterior portion of the eye 1. The light generated by the light source 21 can then be used to illuminate the whole portion of the lens capsule and to record an image of the lens capsule. For this purpose, a two-dimensional detector array 43 is included in the optics portion 3, wherein a camera lens 45 is provided such that an image of the anterior portion of the eye 1 and, in particular, the lens capsule is formed on the detector array 43. The detector array 43 transmits a recorded image to the controller 15, and the controller 15 may process the recorded image. Moreover, the controller 15 can transmit the recorded image or an image derived from the recorded image by image processing to a display apparatus 47. FIG. 1 shows a schematic representation of such recorded image in the display apparatus 47 by lids 49, a sclera 50 and an outer rim 51 of a pupil of the eye. The lens capsule will be visible within the image inside of the pupil 51. The lens capsule is normally transparent to visible light and nearly not visible in the recorded image. However, since a light absorbing agent has been applied to the lens capsule, the lens capsule will be also visible in this image, wherein it can be determined from that image whether a sufficient amount of light absorbing agent has been evenly distributed across the lens capsule. This determination can be performed by the operator of the system 3 or, using image processing software, by the controller 15. If the image analysis reveals that the light absorbing agent has been correctly applied to the lens capsule, the modification of the tissue of the lens capsule can be performed by scanning the focused light beam 19 along the predetermined path on the lens capsule. Apparently, the diverging lens 37 has to be removed from the beam path of the beam 19, and the actuator 33 has to be controlled by the controller 15 such that the focus of the beam 19 follows the predetermined scanning path.

While the diverging of the beam 19 for illumination purposes is achieved by inserting the diverging lens 37 into the beam path, a similar result can be achieved by removing a focusing lens, such as the focusing lens 27 from the beam path of the beam 19. Moreover, the same light source 21 is used both for the tissue modification and the illumination necessary for recording an image. It is, however, also possible to use two different light sources for these two different purposes.

The optical portion 3 further comprises a light attenuating filter 55 which can be positioned by an actuator 57 selectively outside and within the beam path of the beam 19 as indicated by an arrow 58 in FIG. 1. The actuator is controlled by the controller 15. When the filter 55 is positioned within the beam path of the beam 19, it reduces the light intensity of the focus beam 19 such that the focused beam 19 does not modify the tissue of the lens capsule. However, the intensity of the beam 19 incident on the lens capsule generates a sufficient amount of stray light such that a position of the focus of the beam 19 on the lens capsule is visible within the image recorded by the detector array 43. Now, the controller 15 has two modes of operation. One mode is the tissue modifying mode which has been described above and in which the beam 19 is scanned along a desired path in order to modify the tissue of the lens capsule along this path. The second mode of operation is an alignment mode used to correctly align the actually scanned path relative to a desired predetermined path on the lens capsule. In the alignment mode, the controller 15 controls the actuator 57 such that the attenuating filter 55 is located within the beam path of beam 19 such that tissue modification by the focused beam 19 is prevented. The controller then controls the actuator 33 such that the focus of the beam 19 is repeatedly scanned along the scan path at it is currently defined in the controller. The scan path is defined by one or more parameters which have current values stored in a memory of the controller 15. The parameters parameterize the scan path with respect to a lateral extension, i.e. a diameter of the scan path if the scan path is a circle, and a lateral and longitudinal position of the optics portion 3 relative to the eye 1. Therefore, the parameters defining the actual scanning path include the position provided by the actuator 13, the position provided by the actuator 29 and the time dependent positions provided by the actuators 33. The values of these parameters define the actual scan path and can be modified in the alignment mode such that the actual path coincides with the predetermined desired path.

Apart from providing the two different light intensities by positioning the light attenuating filter inside or outside of the beam path of the beam 19, it is also possible to control the light source 21 to selectively emit light with two different intensities.

As mentioned above, the focus of the beam 19 on the lens capsule is visible in the image recorded by the detector array 43. In the alignment mode, the controller 15 controls the actuator 33 such that the focus of the light beam 19 is repeatedly scanned along the actual scan path such that the whole actual scan path is visible within the image. The actual scan path visible in the image is indicated by reference numeral 61 in the schematic image shown on the display apparatus 47. From the image alone it would be difficult for an operator to determine whether the actual scan path 61 coincides with the desired predetermined scan path or not. Therefore, the controller 15 is configured to generate a representation of the desired predetermined scan path in the displayed image. Such representation of the predetermined desired scan path is indicated by reference numeral 63 in the schematic image shown on the display apparatus 47. The representation of the predetermined scan path comprises two adjacent spaced apart lines such that the predetermined scan path is located exactly between the two circular lines. FIG. 1 shows an example in which the actual scan path is displaced relative to the predetermined scan path and has a diameter larger than the predetermined scan path 63. An operator may now change the values of the parameters determining the actual scan path 61 until the actual scan path 61 is visible between the two lines 63 representing the predetermined scan path. The change of parameters can be achieved by input operations via a keyboard or mouse.

Moreover, the adjustment of the parameters defining the actual scan path 61 can be performed by the controller 15 automatically since the actual scan path 61 can be identified by the controller 15 by image analysis.

When the actual scan path 61 sufficiently coincides with the predetermined path 63, the mode of operation of the controller 15 can be changed from the alignment mode to the tissue modifying mode in which the attenuating filter 55 is withdrawn from the beam path of the beam 19 and the focus of the beam 19 is scanned along the actual scan path in order to modify the tissue of the lens capsule.

The optics portion 3 may have a further detector 71 which receives light from the lens capsule of the eye 1. In the illustrated example, a semitransparent mirror 72 is used to supply light originating from the eye 1 to both the detector array 43 and the detector 71. The detector 71 does not provide a position resolution, and the light is supplied to the detector 71 via a band pass filter 73 and a focusing lens 74. The band pass filter 73 is designed such that it allows fluorescent light generated by a fluorescence of the light absorbing agent to traverse the filter 73 whereas other light then the fluorescent light does substantially not traverse the band pass filter 73. An intensity of the light detected by the detector 71 is indicative of an intensity of a current tissue modification achieved by the focused light beam 19. A detection signal of the detector 71 is supplied to the controller 15 which controls parameters of the scanning beam 19 based on this detection signal. The controller may, in particular, control the actuator 29 in order to change a longitudinal position of the beam focus such that it is located on the lens capsule even if the lens capsule is curved. The controller 15 may control the light source 21 in order to adjust the intensity of the emitted light such that the optimal tissue modification is achieved, and the controller may control the actuator 33 in order to increase or decrease the scanning speed along the scan path such that the optimum tissue modification is achieved. Moreover, the controller 15 may stop the scanning if the currently detected intensity is below a threshold, which can be an indication that the desired modification of the tissue has been achieved at the illuminated location since the available amount of dye has been consumed and further illumination would only result in undesired damage of the tissue of the lens capsule or adjacent tissues.

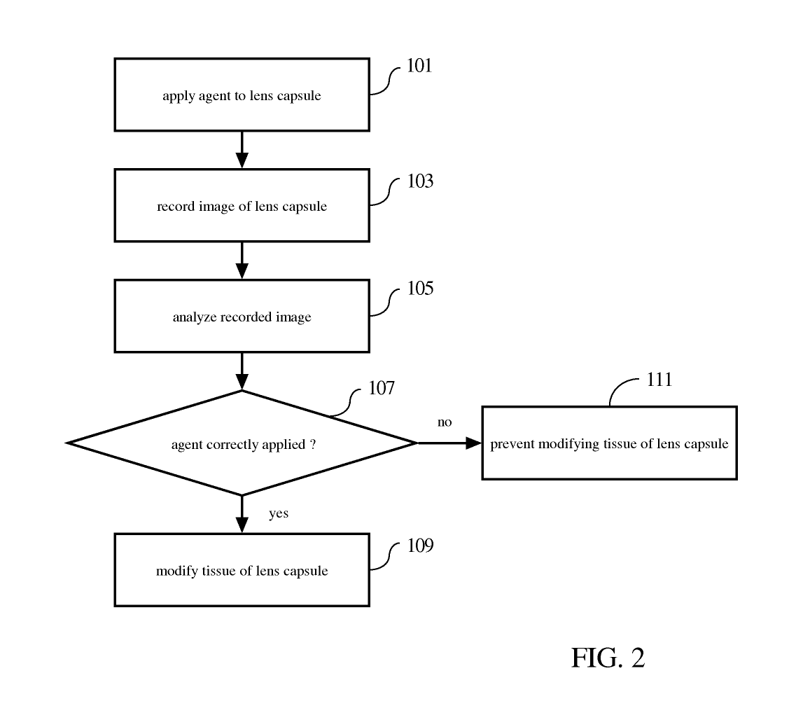

The various functions of the system 3 and methods which can be performed using the system 3 will be summarized with reference to FIGS. 2 to 4 below.

FIG. 2 is a flowchart illustrating an embodiment of a method of performing a capsulotomy. A light absorbing agent is applied to a lens capsule in a step 101. An image of the lens capsule is recorded in a step 103. The recorded image is analyzed in a step 105. In a step 107, it is determined whether the light absorbing agent has been correctly applied to the lens capsule. If the light absorbing agent has been correctly applied to the lens capsule, the tissue of the lens capsule is modified in a step 109 by scanning a focused light beam along a predetermined path. If it is determined in step 107, that the light absorbing agent has not been correctly applied to the lens capsule, the modifying of the tissue of the lens capsule is prevented in a step 111.

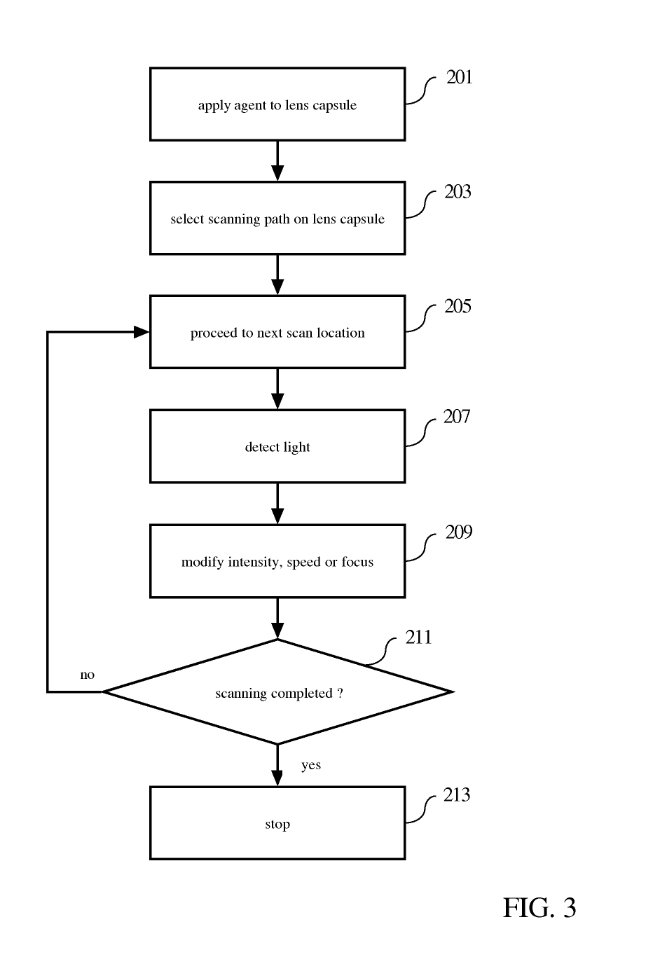

FIG. 3 shows a flowchart of a further embodiment of a method of performing a capsulotomy.

A light absorbing agent is applied to a lens capsule in a step 201. A scanning path on the lens capsule is selected in a step 203. A scanning of a focused light beam along the scanning path selected in step 203 is performed in steps 205, 207, 209 and 211. In step 205, the focus of the light beam proceeds to the next scan location. Light originating from the lens capsule and generated by the incident focused light beam is detected in step 207. The further scanning is controlled based on the light detected in step 207. In particular, the intensity of the incident focused light beam, the scanning speed or the position of the focus of the focused light beam can be modified in step 209 based on the light detected in step 207. It is apparent that the modifying of the intensity, speed or focus in step 209 also include that values of parameters representing the intensity, speed or focus are not changed in the step 209 if such change is not necessary. In step 211 it is determined whether the scanning is completed. If the scanning is not completed, the processing continues with step 205 such that the steps 205, 207, 209 and 211 are repeated until it is determined in step 211 that the scanning is completed such that the procedure can stop at a step 213.

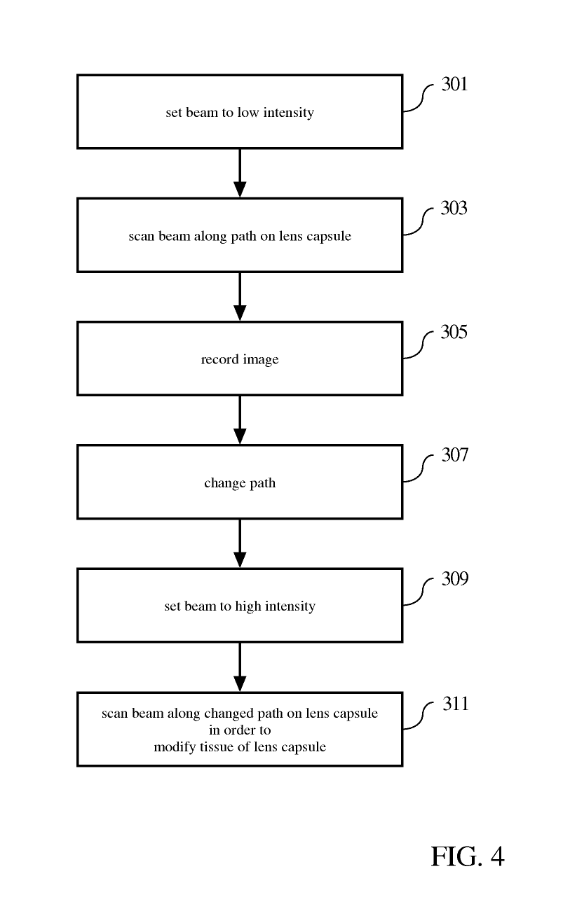

FIG. 4 is a flowchart illustrating a further embodiment of a method of performing a capsulotomy.

The flowchart in particular illustrates an alignment of a scan path relative to a lens capsule. The focused light beam used for modifying the tissue is set to a low intensity in a step 301 such that the light beam may not modify the tissue. The focused light beam is then scanned along an actual scanning path on the lens capsule in a step 303, wherein the actual scan path is defined by values of parameters representing the scan path. An image of the lens capsule is recorded in a step 305, wherein the actual scan path is visible within the image. The values of the parameters representing the scan path are changed in a step 307 based on the image recorded in step 305. In the illustrated example, it is assumed that the actual scan path coincides with the predetermined scan path after the path has been changed in step 307. It is, however, possible to repeat the sequence of steps 303, 305 and 307 until the actual scan path coincides with the predetermined scan path.

If the actual scan path sufficiently coincides with the predetermined scan path, the intensity of the focused light beam is set to the high intensity in a step 309 such that the focused light beam can modify the tissue. The focused light beam is then scanned along the scan path on the lens capsule in order to modify the tissue of the lens capsule in a step 311.

In the above illustrated embodiments, it is also possible to insert a light absorbing agent into the lens capsule such that the light absorbing agent is located behind the tissue which is to be modified, with the intention that this light absorbing agent absorbs light which has traversed the tissue to be modified such that such light will not cause damage to other tissues, such as the retina.

In the embodiments illustrated above, an anterior capsulorhexis is formed in the lens capsule. The natural lens can be removed via the anterior capsulorhexis and the intraocular lens can be inserted via the anterior capsulorhexis. It is, however also possible to form a posterior capsulorhexis after the natural lens has been removed. The anterior capsulorhexis and the posterior capsulorhexis can then be used to hold an intraocular lens at its outer rim as illustrated in U.S. Pat. No. 6,027,531, the full disclosure of which is incorporated herein by reference. Such method is also known as “bag-in-the-lens” method.

In the embodiments illustrated above, it is further desirable that the lens capsule is contacted by air when the light absorbing agent is applied and that the lens capsule is contacted by water or a viscoelastic medium when the predefined line is illuminated for modifying the tissue of the lens capsule. It is possible to use methods of optical coherence tomography or confocal microscopy to verify such conditions.

While the disclosure has been described with respect to certain exemplary embodiments thereof, it is evident that many alternatives, modifications and variations will be apparent to those skilled in the art. Accordingly, the exemplary embodiments of the disclosure set forth herein are intended to be illustrative and not limiting in any way. Various changes may be made without departing from the spirit and scope of the present disclosure as defined in the following claims.

1-46. (canceled)

47. A method of performing a capsulotomy, comprising:
applying a light-absorbing agent to a lens capsule;
illuminating the lens capsule using a first light beam and recording an image of at least a portion of the lens capsule;
analyzing the recorded image and determining whether the light absorbing agent has been correctly applied to the lens capsule; and
if it is determined that the light absorbing agent has been correctly applied to the lens capsule, directing a shaped second light beam onto a predetermined line on the lens capsule in order to modify a structure of a tissue of the lens capsule.

48. The method according to claim 47, further comprising blocking the directing of the shaped second light onto the predetermined line on the lens capsule, if it is determined that the light absorbing agent has not been correctly applied to the lens capsule.

49. The method according to claim 47, wherein the first light beam includes light of a wavelength which is equal to a wavelength of light included in the second light beam.

50. The method according to claim 49, wherein the first light beam and the second light beam are generated by a same light source.

51. The method according to claim 50, wherein the first light beam is generated by defocusing the second light beam.

52. The method according to claim 47, wherein the directing of the shaped second light beam onto the predetermined line on the lens capsule comprises scanning of the second light beam along the predetermined path, wherein the second light beam is a focused light beam.

53. The method according to claim 47, wherein the light-absorbing agent is a dye having an affinity to the tissue of the lens capsule, and
wherein the dye is configured such that a portion of the dye which has formed a compound with the tissue due to its affinity to the tissue can be discriminated from a portion of the dye which has not formed a compound with the tissue based on a fluorescent emission of the dye.

54. The method according to claim 53, wherein the first light beam includes light of a first wavelength range from 400 nm to 600 nm.

55. The method according to claim 53, wherein the image is recorded using light of a second wavelength range from 600 nm to 800 nm.

56. The method according to claim 55, wherein light outside of the second wavelength range contributes to an intensity of the recorded image by less than 20%.

57. The method according to claim 47, wherein the directing of the shaped second light beam onto the predetermined line comprises controlling an intensity of the second light beam based on the recorded image.

58. The method according to claim 57, wherein the intensity of the second light beam at a given location along the predetermined path is determined based on an image intensity at a location in the recorded image which corresponds to the given location.

59. A method of performing a capsulotomy, comprising:
applying a light-absorbing agent to a lens capsule;
scanning a focused light beam along a predetermined path on the lens capsule in order to modify a structure of a tissue of the lens capsule;
detecting light of a predetermined wavelength range originating from the lens capsule during the scanning; and
controlling at least one of an intensity of the focused light beam, a scanning speed of the focused light beam along the predetermined path and a focus position of the focused light beam in a longitudinal direction of the focused light beam based on an intensity of the detected light.

60. The method according to claim 59, wherein the controlling of the intensity of the focused light beam includes reducing the intensity of the focused light beam when the intensity of the detected light increases.

61. The method according to claim 59, wherein the controlling of the intensity of the focused light beam includes preventing the focused light beam to reach the lens capsule when the intensity of the detected light is below a predefined threshold.

62. The method according to claim 59, wherein the controlling of the scanning speed of the focused light beam includes increasing the scanning speed of the focused light beam when the intensity of the detected light increases.

63. The method according to claim 59, wherein light-absorbing agent is a dye having an affinity to the tissue of the lens capsule, and
wherein the dye is configured such that a portion of the dye which has formed a compound with the tissue due to its affinity to the tissue can be discriminated from a portion of the dye which has not formed a compound with the tissue based on a fluorescent emission of the dye.

64. The method according to claim 59, wherein the focused light beam includes light of a first wavelength range from 400 nm to 600 nm.

65. The method according to claim 59, wherein the predetermined wavelength range ranges from 600 nm to 800 nm.

66. The method according to claim 65, wherein light outside of the predetermined wavelength range contributes to intensity of the detected light by less than 20%.

67. A method of performing a capsulotomy, comprising:
applying a light-absorbing agent to a lens capsule;
using beam supplying optics to generate a shaped light beam of a first intensity and to direct the shaped light beam of the first intensity onto a selected line positioned relative to the beam supplying optics such that the shaped light beam of the first intensity is incident on an anterior portion of an eye;
generating an image of the anterior portion of the eye wherein a line illuminated by the shaped light beam of the first intensity is detectable based on the generated image;
at least one of positioning the beam supplying optics relative to the eye and modifying a parameter of the shaped light beam based on the detected image such that the illuminated line coincides with a predetermined line on the lens capsule; and then
using the beam supplying optics to generate a shaped light beam of a second intensity greater than the first intensity and to direct the modified shaped light beam of the second intensity onto the selected line positioned relative to the beam supplying optics in order to modify a structure of a tissue of the lens capsule along the predetermined line on the lens capsule.

68. The method according to claim 67, wherein the parameter of the selected line includes at least one of a lateral position of the selected line relative to the beam supplying optics and a lateral extension of the selected line.

69. The method according to claim 67, wherein the directing of the shaped light beam onto the predetermined line on the lens capsule comprises scanning of the shaped light beam along the predetermined line, wherein the shaped light beam is a focused light beam.

70. The method according to claim 67, further comprising generating of a representation of the predetermined line in the generated image.

71. The method according to claim 70, wherein the generating of the representation of the predetermined line in the generated image includes generating a pair of adjacent spaced apart lines such that the predetermined line extends in between the pair of adjacent spaced apart lines.

72. The method according to claim 67, wherein the first intensity is selected such that the shaped light beam of the first intensity does substantially not modify the tissue of the lens capsule.
